(19) United States
(12) Patent Application Publication
(10) Pub. No.: US 2017/0119986 A1
(43) Pub. Date: May 4, 2017

(54) Face Mask for Inhalation, Suitable for Delivering a Therapeutic Gas or Molecule As part of A Treatment By Inhalation, In Particular In Children

(71) Applicant: PROTECSOM AMERIQUE DU NORD INC., Drummondville, Quebec (CA)

(72) Inventor: Thierry Poree, St-Pierre-Eglise (FR)

(21) Appl. No.: 15/122,487

(22) PCT Filed: Jan. 30, 2015
(86) PCT No.: PCT/CA2015/000057

§ 371 Date: Aug. 30, 2016

(30) Foreign Application Priority Data
Mar. 4, 2014 (FR) 1451756

(51) Int. Cl. A61M 16/06; A61M 16/08; A61M 16/20; A61M 11/06; A61M 15/00

(52) U.S. Cl. CPC A61M 16/0616; A61M 11/06; A61M 15/0086; A61M 16/208; A61M 16/0816; A61M 2240/00; A61M 2205/0216; A61M 2210/0618; A61M 2210/0625

(57) ABSTRACT

The present invention relates to a face mask for inhalation, suitable for delivering a therapeutic gas or molecule as part of a treatment by inhalation. In particular, the invention relates to a mask (M) including a body (C), a wall (P1, P2, P3) of which defines an inner space (Vi) intended for engaging with a portion of the face of a user, including the nose and mouth thereof, the body (C) having a first opening (O1) which is in communication with a means (R1) for connecting to a source of a therapeutic gas or molecule, and a second opening (O2) surrounded by a lip (L1) intended to be placed against said portion of the face of the user, characterised in that said mask comprises a sealing partition (C1) which defines, in the inner space (Vi), an upper chamber (B1) intended for accommodating the nose of the user and a lower chamber (B2) containing said first opening (O1) and intended for accommodating the mouth of the user while said mask is being worn.

### RELATED APPLICATIONS

This application claims priority and benefit from patent application FR1451756, filed Mar. 4, 2014.

### BACKGROUND

Field of Invention

The present invention relates to a face mask for inhalation, adapted for administering a gas or a therapeutic molecule during an inhalation treatment, particularly for children.

Description of Prior Art

A face mask for inhalation is a medical device that improves the intake of a breathing gas in the body of a person who has a temporary or permanent difficulty with their respiratory reflex. It can also be used to diffuse a therapeutic molecule into the body of a patient via their respiratory tract.

The respiratory tract extends from the nose and mouth down to the pulmonary alveoli. They are responsible for an individual's breathing function and are therefore necessary for living. Intake of breathing gas takes place at the nose and/or mouth level, while the pulmonary alveoli are where gaseous exchanges with the inside of the human body take place. This is also where dioxygen is absorbed into the body, while carbon dioxide is rejected into the atmosphere.

Lungs contain all of the pulmonary alveoli. To ensure their function, these organs exhibit a particularly large exchange surface with inner medium. This particularity makes lungs the preferred location for absorption of therapeutic molecules.

The nose is lined with mucosa rich in blood vessels. The mucosa is covered with nasal mucus, which traps the incoming molecules and allows contact with nerve endings for the purpose of olfaction. There is also provided a ciliary system in the nose, whose role is to filter the inhaled air to avoid microorganisms or foreign molecules.

It should be understood that the nose is a barrier against the passage of therapeutic molecules to the lungs, and therefore inhalation of a drug should be made preferably by mouth breathing. However, nose breathing is a reflex, so it is hard to force the patient to breathe through the mouth only, especially when the patient is a child or a baby.

For example, a traditional face mask for inhalation such as the one described in the patent application EP 1,389,482 is an oral nasal breathing mask, that surrounds both the mouth and nose when applied to the patient's face. It comprises a single cavity and an opening for communication with an element on the mask such as a source of O₂. This mask makes it possible to improve the patient's breathing, but is not well adapted for inhalation of therapeutic molecules because they cannot be preferentially directed toward one or other of the respiratory system entries. Patent application WO 2012/173,992 discloses a face mask for inhalation having a portion housing the nose and a portion housing the mouth of the patient, and an opening aligned with the nose for inhalation of therapeutic molecules by the patient. This solution favors the inhalation of therapeutic molecules through the nose, which is an obstacle to an optimal deposition of therapeutic molecules in the patient's lungs.

Patent application US 2005/217 678 describes a face mask for inhalation with an intraoral member. The constraint of mouth breathing is thereby ensured, but the intraoral member is a real discomfort for the patient, and is particularly unsuitable when the patient is an infant.

CN 203090169 U utility patent application has a breathing mask to reassure the child and to administer medications. The mask comprises a pipe extending into the patient's mouth cavity; however it is not a very comfortable solution, especially for children and infants.

In the current state of the art, there are thus inhalation masks forcing the patient to mouth breathe via intraoral members. These devices are nevertheless uncomfortable for the patient and especially unsuitable for young patients, hampering drug inhalation and causing discomfort to the patient. Moreover, such devices are complex to manufacture and/or to use.

The object of the invention is to overcome the aforementioned drawbacks. In particular, an object of the invention is to provide a face mask for inhalation designed to permit optimal inhalation and lung deposition of therapeutic molecules without necessitating significant constraints or causing discomfort when the patient uses the mask.

In particular, an object of the invention is to provide such a mask for mouth-only inhalation of the gas or therapeutic molecule.

Another object of the invention is to provide such a mask in order to reduce the risk of the patient refusing to use the mask.

Another object of the invention is to provide such a mask that has a simple design and is simple to use.

### SUMMARY

To this end, the present invention provides a mask comprising a body including at least one wall defining an inner space for engaging a portion of a user's face including the nose and mouth, the body having a first opening in communication with a connection means to a source of a gas or a therapeutic molecule, and a second opening bounded by a lip to be applied against said portion of the user's face, characterized in that it comprises a partition defining, in the inner space, an upper chamber for accommodating the nose of the wearer and a lower chamber containing said first opening and for accommodating the mouth of the user when said mask is being worn.

The mask covers both the nose and mouth of the patient. The partition makes an airtight separation between the chamber receiving the nose and the chamber receiving the mouth of the user. Only the mouth-receiving chamber allows communication of the respiratory system of the user with the source generating the respiratory gas or therapeutic molecule via the connecting means R1. An individual's inhalation is thus forced only through the mouth. According to one embodiment of the invention, the partition is bordered by a second lip to be applied above the upper lip of the user. Advantageously, the second lip merges at its ends with the first lip.

The lips of the mask thus joined ensure the sealing of the labial edge and nose. This feature enhances the seal between the two chambers and helps force the inhalation of the gas or the therapeutic molecule by mouth. Preferably, the mask consists of a single ergonomically molded piece for example in an injection molding process. According to one embodiment of the invention, the upper chamber includes a unidirectional expiratory valve.

The unidirectional expiratory valve is designed to allow the exit of air exhaled by the patient, to prevent the entry of air in this part of the mask and is provided to improve patient comfort. The valve favors the patient's inspiration through the mouth during the breathing effort.

According to an embodiment of the invention, the lip has an overall trilobic shape.

The mask with a trilobic cross-section is specifically adapted for very young children, since the mouth-nose distance is very small for this type of patient. In particular, this variant of the mask design is used to limit direct gas exchanges between the nose chamber and ambient air.

According to an embodiment of the invention, the lip has an overall piriform shape.

The mask with a pear-shaped lip is particularly suitable for adults and children. Indeed, these people have particular morphological features in the oro-nasal area, particularly with regard to the mouth-nose distance, but this distance is greater than that observed for infants and very young children. This type of mask therefore makes it possible to take into account the requirements of the user's face, including ensuring the tightness of contact between the lips of the mask and the face.

According to an embodiment of the invention, the two chambers are linked by connecting means.

For example, the body of the mask can be split in such a way that both chambers are disjoint, for example, for easy storage. In such a case, fastening means ensure the unity of the mask.

According to one embodiment of the invention, the mask is made of a plastic material or an elastomer material.

When the plastic material is an elastomer, the use of a silicone-based material is preferred. Silicone has the advantage to be as neutral as possible from a biological point of view, making it an ideal material for the manufacture of an element intended for contact with the human body. There are nevertheless on the market synthetic elastomers that also have all the features necessary for the design of such a mask. Such elastomers are of course contemplated within the scope of the present invention.

The invention also relates to an inhalation device as described above comprising an inhalation mask as described above and an inhalation chamber or a nebulizer, the inhalation chamber and the nebulizer being connected to the connection means of the mask.

The present invention aims to provide a mask for administering a gas or a therapeutic molecule, comprising a single molded piece for engaging the portion of a user's face including the nose and mouth, said piece comprising a body of which at least one wall defines an inner space, the body comprising an airtight partition delimiting, in the inner space, an upper chamber for receiving the nose of the user and a lower chamber for receiving the mouth of the user when said mask is being worn the bottom chamber, the body having a first opening in communication with a connection means to a source of a gas or a therapeutic molecule, the connection means opening into the lower chamber without penetrating into the mouth, and a second opening bordered by a lip curved toward the inside of the body, to be applied against said portion of the user's face.

The inhalation mask according to the present invention may be characterized in that the partition is bordered by a second lip to be applied above the upper lip of the user.

The inhalation mask according to the present invention may be characterized in that the second lip merges at its ends with the first lip.

The inhalation mask according to the present invention may be characterized in that the upper chamber comprises a unidirectional expiratory valve.

The inhalation mask according to the present invention may be characterized in that the lip bordering said second opening to be applied against said portion of the user has an overall trilobic shape.

The inhalation mask according to the present invention may be characterized in that the lip bordering said second opening to be applied against said portion of the user face has an overall piriform shape.

The mask according to the present invention may be characterized in that it is made of a plastic material or an elastomer material.

The present invention aims to present, in a further aspect of the invention, an inhalation device, characterized in that it comprises an inhalation mask according to the present invention and an inhalation chamber or nebulizer, the inhalation chamber being connected to the connection means.

Aforementioned features of the invention, as well as others, will become more apparent in light of the following detailed description of an exemplary embodiment.

### BRIEF DESCRIPTION OF THE DRAWINGS

The invention will be better understood by referring to the accompanying figures, which are to be regarded as illustrative in nature, and not as limiting the scope of the invention. In the figures:

FIG. 1 shows a perspective front view of a face mask for inhalation according to an embodiment of the invention;

FIG. 2 shows a rear perspective view of a face mask for inhalation according to an embodiment of the invention;

FIG. 3 shows a cross section view of a face mask for inhalation according to an embodiment of the invention;

FIG. 4 shows a rear view of a face mask for inhalation according to an embodiment of the invention;

FIG. 5 shows a rear view of another embodiment of a face mask for inhalation suitable for the face shape of an infant, according to an embodiment of the invention;

FIGS. 6 and 7 show two rear views of two inhalation mask according to FIGS. 4 and 5, comprising an exhalation valve;

FIG. 8 shows a view of a face mask for inhalation according to an embodiment of the invention, coupled to an inhalation chamber.

It will be noted that throughout the appended drawings, like features are identified by like reference numerals.

### DETAILED DESCRIPTION

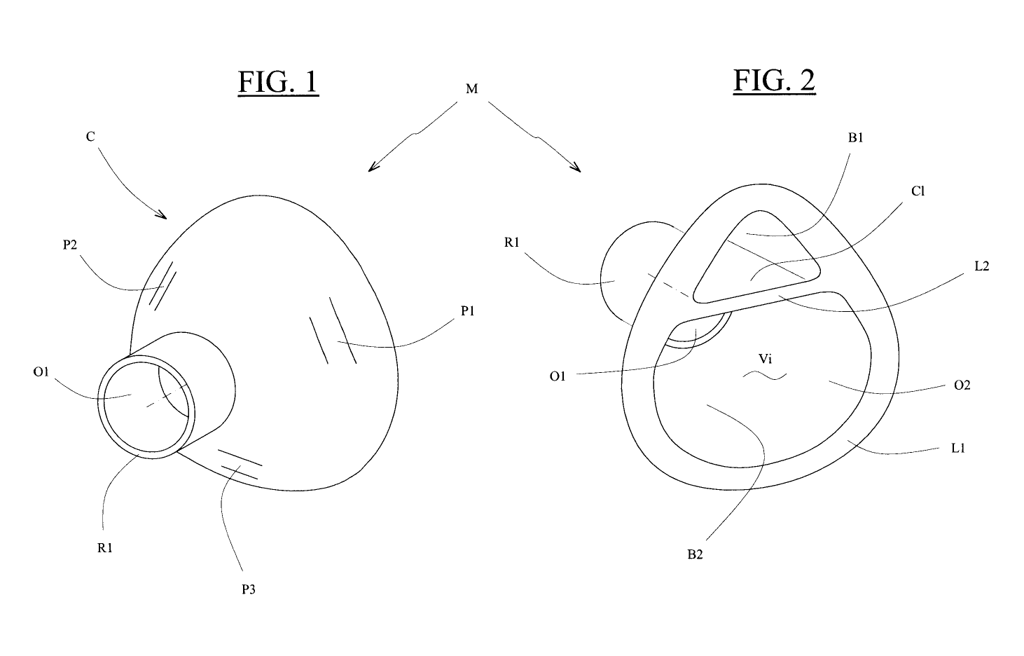

The invention presented in FIGS. 1 to 7 relates to a mask (M) comprising a body (C) of which at least one wall (P1, P2, P3) defines an internal volume (Vi) for engaging a portion of a user's face including the nose and mouth, the body (C) comprising a first opening (O1) in communication with a connection means (R1) to a source of gas or therapeutic molecule, and a second opening (O2) bordered by a lip (L1) intended to be applied against said portion of the user's face, characterized in that it comprises an airtight partition (C1) defining, in the internal volume (Vi), an upper chamber (B1) for receiving the nose of the user and a lower chamber (B2) containing said first opening (O1) and for housing the mouth of the user when said mask is being worn.

This is a face mask for inhalation, for administering a gas or a therapeutic molecule in the context of an inhalation therapy. The face mask for inhalation according to the invention is, in particular but in a non-limiting manner, intended for use by a child, an infant and/or a preterm baby.

In FIGS. 1 to 7, the face mask for inhalation M comprises a body C having a space Vi which opens up, at an opening O1, on a connection means R1 connecting to a device such as an inhaler, a nebulizer or any other source generating a gas or an airborne therapeutic molecule.

In the embodiment shown in FIGS. 1, 2, 3 and 4, the body C has the overall shape of a pyramid section with three faces and curved edges. It is thus generally defined by three walls P1, P2 and P3. This embodiment is particularly relevant for making a mask, according to the invention, that meets the morphological requirements of an adult or a child.

In the embodiments shown in FIGS. 5 and 7, the body C has the overall shape of a sphere section. It is thus generally defined by three identical walls P1, P2 and P3. This embodiment is particularly relevant for making a mask which meets the morphological requirements of an infant or preterm baby.

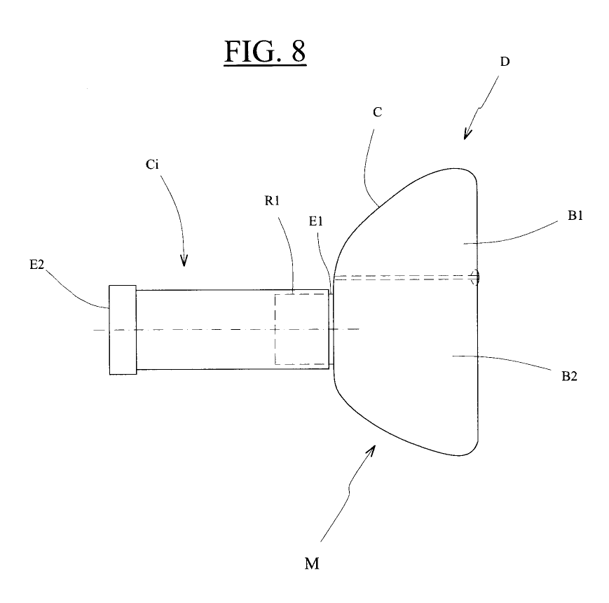

In these embodiments, the connecting means R1 is a hollow portion of cylinder that makes the mask communicate with a complementary device such as an inhalation chamber, as shown in FIG. 8, or a nebulizer.

As shown in FIG. 2, the body C is delimited by an opening O2 bordered by a lip L1 to be applied around the oronasal area of the user. The lip L1 is bent toward the inside of the body C of the mask M, so that the free edge of the lip L1 is not in contact with the mask user.

In a rear view, as in FIG. 4, the lip L1 of the face mask M for inhalation has a piriform shape. It is particularly suited to children whose mouth-nose distance begins to increase significantly.

In another embodiment, FIG. 5 shows a version of the mask that is suitable for users whose nose-mouth distance is smaller, i.e., suitable for infants and preterm babies. In a rear view, as in FIG. 5, the lip L1 of this mask has a trilobic shape.

There is shown in FIGS. 2 to 7 a partition C1 delimiting, inside the body C of the mask M, two chambers: an upper chamber B1 and a lower chamber B2. The upper chamber B1 is for receiving the nose of the user while the lower chamber B2 is for receiving the mouth of the user when the mask M is being worn. The partition C1 extends, as seen in FIGS. 2 and 4 to 7, over the entire width of the mask, of the wall P1 to the wall P2.

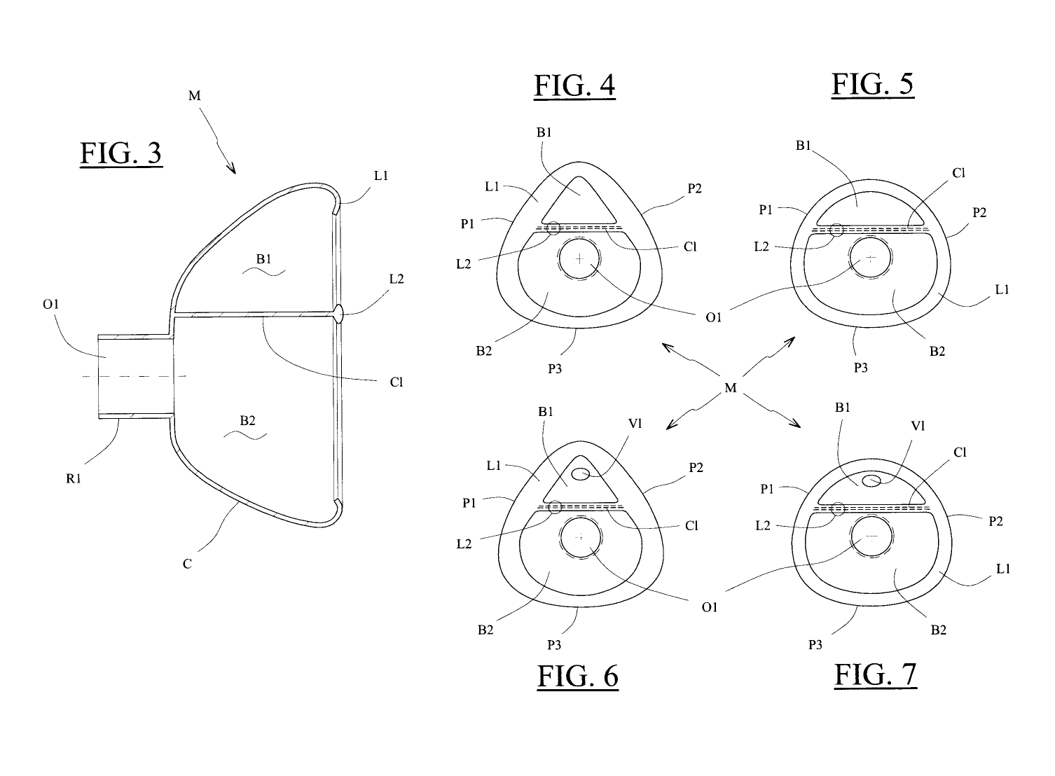

It can be seen in FIG. 3 that the partition C1 also extends over the entire depth of the mask. When the mask is being installed on a standing person, the partition C1 is connected above the connecting means R1.

The free edge of the partition C1 is delimited by a second lip L2 (FIG. 3). L2 lip is to be brought into contact with the user's face and precisely above the upper lip, and offset of the nostrils.

It is also shown in FIG. 3 that the second lip L2 is connected at its two ends with the lip L1. Accordingly, lips L1 and L2 are continuous. This provides an airtight seal on the lip contour and around the nose. The upper chamber B1 houses only the nose, while the lower chamber B2 houses only the mouth of the mask's user. The chambers B1 and B2 have an airtight separation between them.

The connection means R1 communicates with the mouth chamber B2 of the face mask M for inhalation. The user is thus forced to inhale by the mouth only. Inhalation is performed without tubes to be inserted into the mouth. The use of the mask M is particularly easy, especially for nursing young children and infants.

According to another embodiment shown in FIGS. 6 and 7, an expiratory valve V1 is provided at an opening in the upper chamber or nose chamber B1. This is a unidirectional valve which is located at a curved wall making the junction between the wall P1 and the wall P2. It allows the user to expel the air in their lungs out of the mask, without discomfort.

The mask according to the invention causes no discomfort during use, especially in the oral cavity.

It reduces the risk of a patient refusing to use the mask.

It facilitates its use by caregivers, both professional and non-professional.

It should be noted that the mask according to the invention may comprise of two disjoint chambers, the two chambers being linked by fastening means (embodiment not shown).

In FIG. 8, there is shown an inhalation device D. A face mask M for inhalation, such as one of those described in reference with FIGS. 1 to 7, is connected via its connecting means R1 to an inhalation chamber Ci. A source that generates a gas or a therapeutic molecule, such as an aerosol dispenser, can be connected to the second end E2 of this inhalation chamber to allow the molecule or gas to diffuse within the inhalation chamber Ci during treatment and after actuating the aerosol dispenser. During the patient-triggered inspiration/inhalation phase, the therapeutic molecule will diffuse from the inhalation chamber Ci to the lower chamber B2 of the mask M via the R1 connecting means. The partition C1 will prevent the therapeutic molecule or the gas from reaching the upper chamber B1, thus preventing inhalation of the therapeutic molecule or gas by any way other than the user's mouth.

While preferred embodiments have been described above, it will be evident that modifications may be made without departing from the scope of the disclosure.

| Reference | Description | FIG. |
|---|---|---|
| M | Face mask for inhalation | 1 2 3 4 5 6 7 8 |
| C | Body | 1 3 8 |
| R1 | Connection means (tube) | 1 2 3 8 |
| P1 P2 P3 | Walls | 1 4 5 6 7 |
| L1 | Lip | 2 3 4 5 6 7 |
| L2 | Second lip | 2 3 4 5 6 7 |
| C1 | Partition | 2 3 4 5 6 7 |
| B1 B2 | Chambers | 2 3 4 5 6 7 8 |
| V1 | Expiratory valve | 6 7 |
| Ci | Inhalation chamber | 8 |
| Vi | Inner space | 2 |
| O1 | First opening | 1 2 3 4 5 6 7 |
| O2 | Second opening | 2 |
| D | Inhalation device | 8 |

1. A mask (M) for administering a gas or a therapeutic molecule, comprising a single molded piece for engaging a portion of a user's face including user's nose and mouth, the piece comprising a body (C) of which at least one wall (P1, P2, P3) defines an internal volume (Vi), the body (C) comprising a partition (C1) defining, in the inner space (Vi), an upper chamber (B1) for receiving the nose of the user, the upper chamber (B1) comprising a unidirectional expiratory valve (V1), and a lower chamber (B2) for receiving the mouth of the user when the mask is being worn, the body (C) having a first opening (O1) in communication with a connection means (R1) to a source of a gas or a therapeutic molecule, the connection means (R1) opening into the lower chamber (B2) without penetrating into the mouth, and a second opening (O2) bordered by a lip (L1) curved toward inside the body (C) and to be applied against the portion of the user's face.

2. The mask of claim 1, wherein the partition (C1) is bordered by a second lip (L2) to be applied above the upper lip of the user.

3. The mask of claim 2, wherein the second lip (L2) merges at its ends with the first lip (L1).

4. (canceled)

5. The mask of claim 1, wherein the lip (L1), bordering the second opening (O2) to be applied against the portion of the user's face, has an overall trilobic shape.

6. The mask of claim 1, wherein the lip (L1), bordering the second opening (O2) to be applied against the portion of the user's face, has an overall piriform shape.

7. The mask of claim 1, wherein the mask is made of a plastic material or an elastomer material.

8. An inhalation device, comprising the mask of claim 1 and an inhalation chamber (Ci) or a nebulizer, wherein the inhalation chamber is connected to the connection means (R1).
